(19) United States
(12) Patent Application Publication
(10) Pub. No.: US 2026/0363749 A1
(43) Pub. Date: Nov. 19, 2026

(54) ROD BENDER POSITIONING MECHANISM

(71) Applicant: GLOBUS MEDICAL, INC., AUDUBON, PA (US)

(72) Inventors: Timothy James, Hatboro, PA (US); Vishnu Kolal, King of Prussia, PA (US); George Yacoub, Lansdale, PA (US); David Leff, Philadelphia, PA (US); Matthew Bechtel, Philadelphia, PA (US)

(21) Appl. No.: 19/085,260

(22) Filed: Mar. 20, 2025

(51) Int. Cl. A61B 17/88; A61B 17/70

(52) U.S. Cl. CPC A61B 17/8863; A61B 17/7013

(57) ABSTRACT

A rod bending system that includes a cart, and a sterile cartridge disposed on the cart that includes a bender configured to bend a rod, a positioner, a carriage configured to hold the rod, a rotate shaft coupled to the carriage and the positioner, and a feed screw coupled to the carriage and the positioner.

The present disclosure relates to medical devices, and more particularly, surgical systems for bending surgical rods, and related methods and devices.

### BACKGROUND

Spinal fusion is a surgical procedure used to correct deformity of the spine by fusing together the painful part of the spine in order to restrict its motion and relieve painful symptoms. Spinal fusion surgery is commonly utilized to treat abnormal spinal curvatures, such as scoliosis and abnormal kyphosis, for example, degenerative disc diseases, spondylolisthesis, trauma resulting in spinal nerve compression, vertebral instability caused by infections or tumors, and other conditions.

Fusion surgery may include the placement of rods and screws using instrumentation and/or the placement of bone graft in between the vertebrae. During surgery, the surgeon may correct the deformity of the spine so as to ensure that the radiographic parameters of the spine in both the sagittal and coronal plane fall within clinically accepted values. While doing so, the surgeon fixes the corrected spine into place using metallic rods. The rods need to conform to the shape of the spine and hence need to be bent accordingly.

Currently, devices such as French bender and power bender are utilized in the operation room in order to bend the rods to the desired curvature. However, these devices require cumbersome manual processes to operate. In addition, use of these devices to bend the rod may also introduce notches on the rod, which may decrease the rod's fatigue life.

### SUMMARY

These drawings illustrate certain aspects of some examples of the present disclosure and should not be used to limit or define the disclosure.

According to some other embodiments of inventive concepts, a rod bending system may include a first lock collar that includes a first linear lock, and a first rotation lock, a second lock collar that includes a second linear lock, and a bender configured to bend a rod.

According to some other embodiments of inventive concepts, a rod bending system may include a cart, and a sterile cartridge disposed on the cart that includes a bender configured to bend a rod, a positioner, a carriage configured to hold the rod, a rotate shaft coupled to the carriage and the positioner, and a feed screw coupled to the carriage and the positioner.

### BRIEF DESCRIPTION OF DRAWINGS

These drawings illustrate certain aspects of some examples of the present disclosure and should not be used to limit or define the disclosure.

FIG. 1A is a diagram showing a lock collar on a rod.

FIG. 1B is a diagram showing two lock collars, installed in opposite directions, on a rod.

FIG. 1C is a diagram showing two lock collars used for controlling the translation and rotation on a rod.

FIG. 2A is a cutaway diagram showing an exploded view of a linear lock and the components thereof.

FIG. 2B is a cutaway diagram showing an assembled view of a linear lock and the components thereof.

FIG. 3A is a diagram of a rod approaching a linear lock.

FIG. 3B is a diagram of a rod entering a linear lock.

FIG. 3C is a diagram of a rod displacing ball bearings and shifting the cage.

FIG. 3D is a diagram of a rod fully traversed through a linear lock.

FIG. 4A is a diagram showing a rod attempting to be removed from a linear lock.

FIG. 4B is a diagram showing a closeup of the rod interacting with a ball bearing during rod removal.

FIG. 5A is a cutaway diagram showing the components of a rotation lock.

FIG. 5B is a cutaway diagram showing the components of a rotation lock with a rod inserted therethrough.

FIG. 6A is a diagram showing a rod attempting to rotate in a rotation lock.

FIG. 6B is a diagram showing a closeup of the rod interacting with a ball bearing during rod rotation.

FIG. 7A is a diagram showing a rod bending system and the components thereof.

FIG. 7B is a diagram showing a sterile cartridge and the components thereof.

FIG. 8A is a diagram showing an exploded view of a positioner and the components thereof.

FIG. 8B is a diagram showing a button and surrounding components of a positioner.

FIG. 8C is a cutaway diagram showing a positioner and the components thereof.

FIG. 8D is a diagram showing a mandrel.

FIG. 9A is a side cutaway diagram showing a carriage and the components thereof.

FIG. 9B is a front cutaway diagram showing a carriage and the components thereof.

FIG. 10 is a diagram of an example computing environment.

FIG. 11 is a flowchart of a method for assembling a rod bending system and bending a rod.

### DETAILED DESCRIPTION

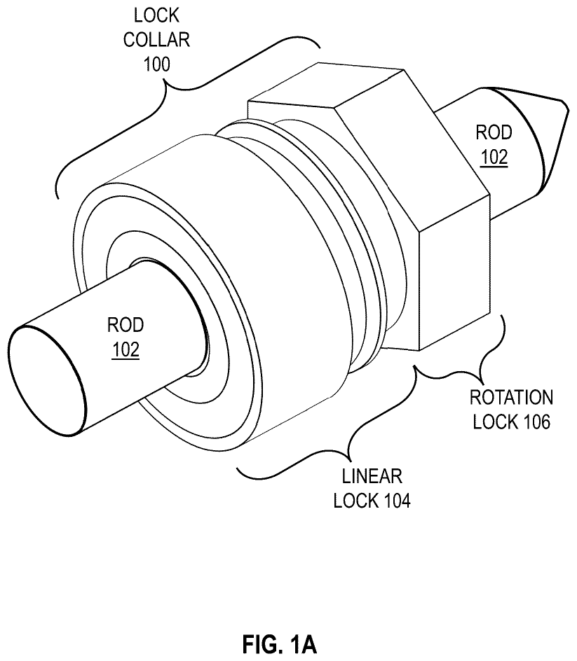

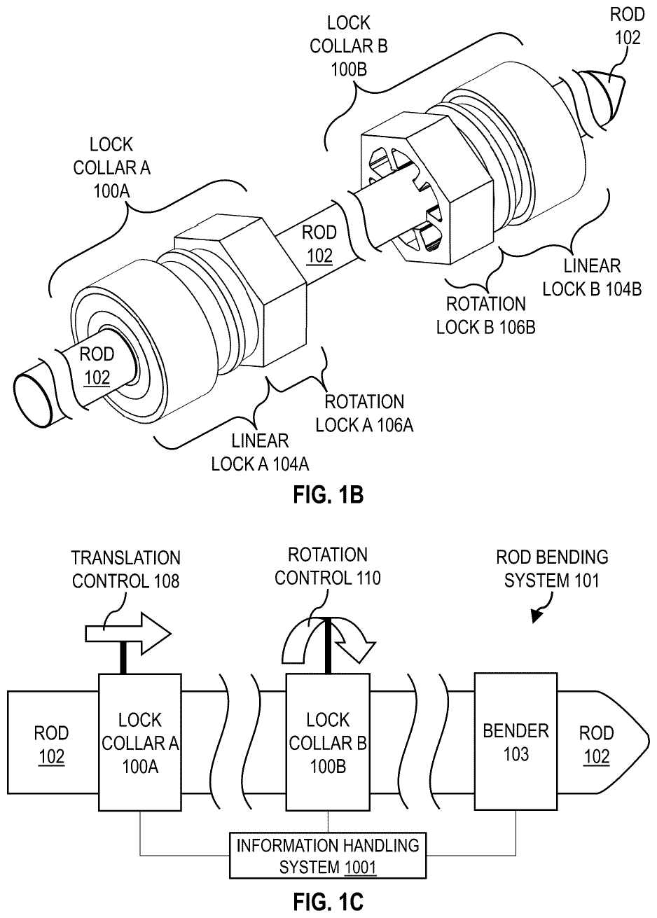

### FIGS. 1A-1C

FIG. 1A is a diagram showing a lock collar on a rod. FIG. 1B is a diagram showing two lock collars, installed in opposite directions, on a rod. FIG. 1C is a diagram showing two lock collars used for controlling the translation and rotation on a rod.

Lock collar 100 is mechanical device which is a combination of one or more linear locks 104 and/or one or more rotation locks 106. In one or more embodiments, lock collar 100 may be used to constrain or control linear and/or rotation of rod 102. Additional details for the function of lock collar 100 may be found in the description of linear lock 104 and rotation lock 106.

Rod 102 is an elongated structure which is generally rigid. In one or more embodiments, rod 102 may be made from one or more metals, polymers, or composite materials and may be coated with one or more different materials. The cross-section of rod 102 may be circular, rectangular, and/or any suitable shape for the desired use. Rod 102 may have a “pointed” end generally formed by a tapered outer surface with a narrowing diameter along longitudinal axis 224 (e.g., a conical shape). Rod 102 may be bent by a rod bending system 700 intraoperatively. In any embodiment, rod 102 may be bent to a desired shape with one or more curves throughout and cut to a desired length. Rod 102 may be used in spinal fusion surgery and implanted into a patent.

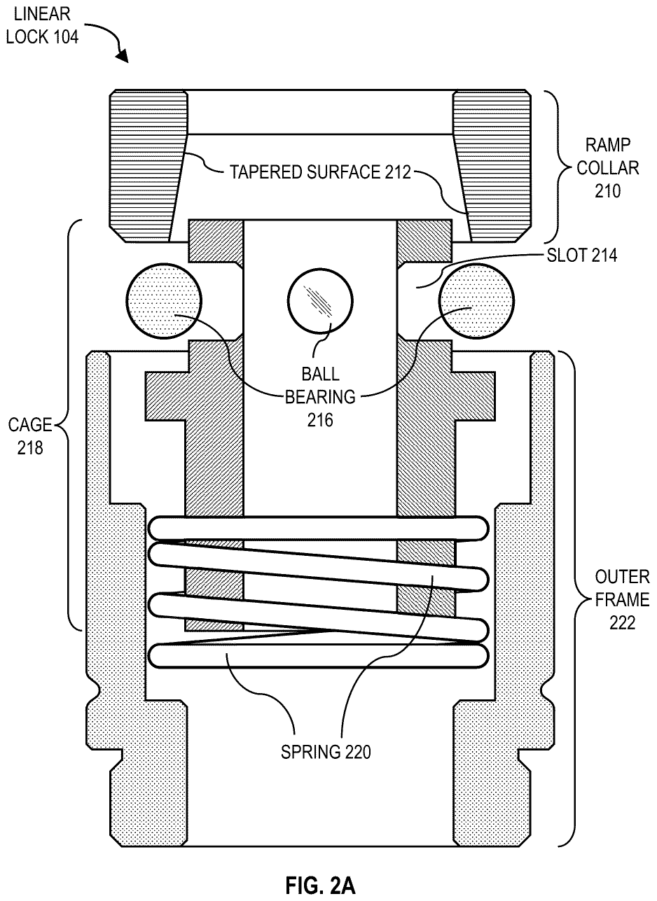

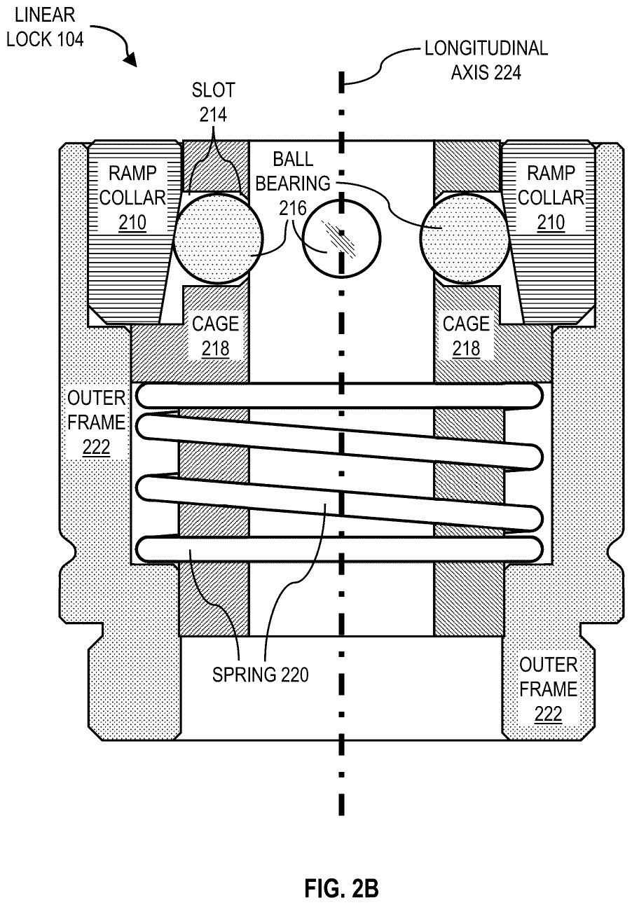

Linear lock 104 is a mechanical device configured to permit movement of rod 102 in a single direction along longitudinal axis 224 while preventing movement in an opposing direction along longitudinal axis 224. In one or more embodiments, linear lock 104 may include an opening, through hole, aperture, and/or other path along longitudinal axis 224 to allow for rod 102 to be disposed therethrough. In one or more embodiments, linear lock 104 may include a tapered opening to guide rod 102 into the center of linear lock 104 (and all of the components therein). Linear lock 104 may include ball bearings 216 and tapered surfaces 212 that engage with a surface of rod 102, thereby allowing movement of rod 102 in one direction while restricting movement of rod 102 in the reverse direction. Additional details regarding linear lock 104 may be found in the description of FIGS. 2A-2B, 3A-3D, and 4A-4B. In other embodiments, it is contemplated to use a cylindrical roller/pin rather than ball bearings.

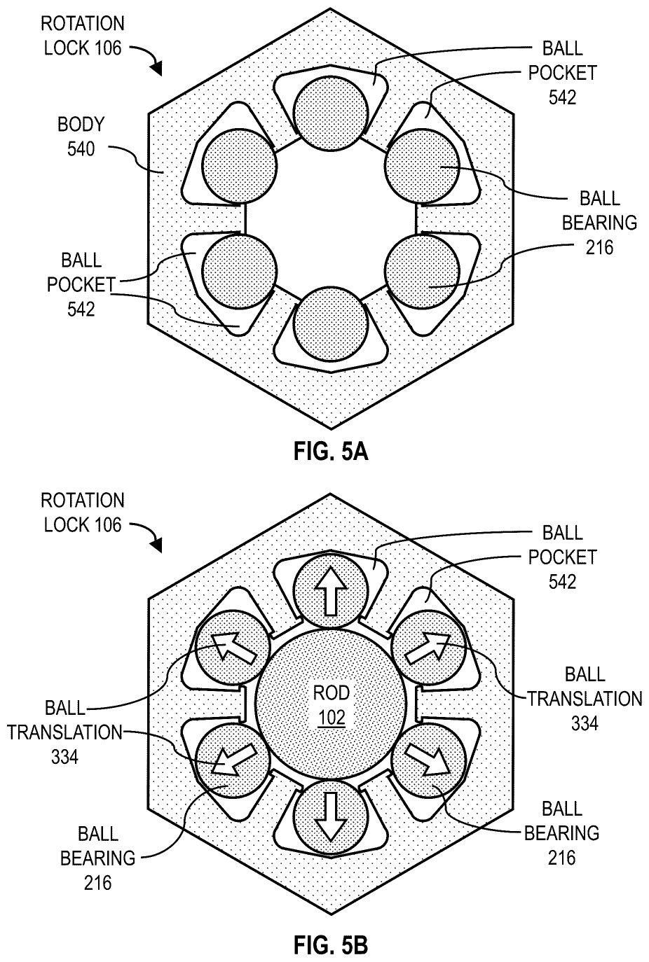

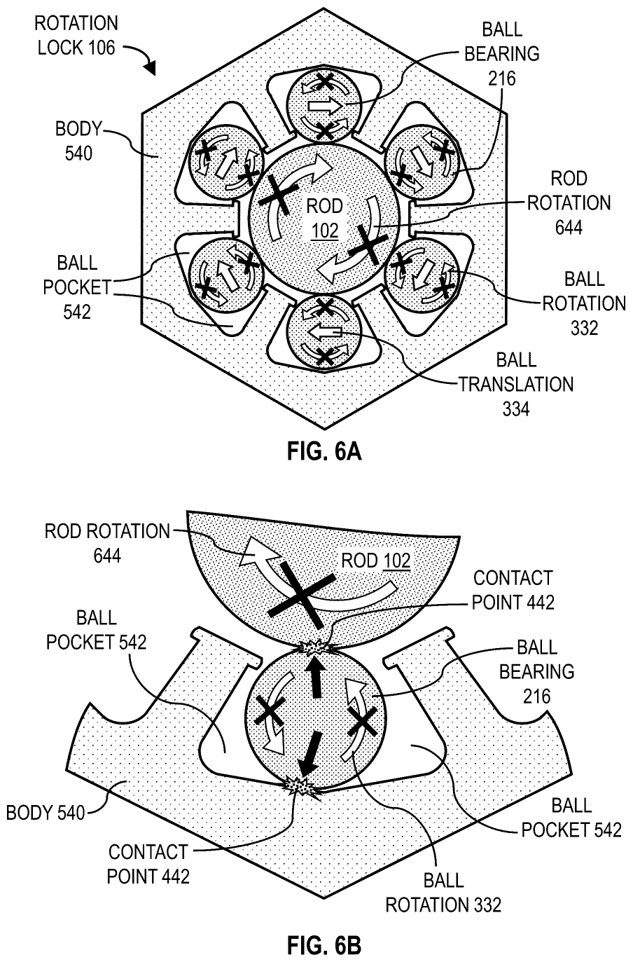

Rotation lock 106 is a mechanical device configured to limit rotation of rod 102 with respect to rotation lock 106. In one or more embodiments, rotation lock 106 may include an opening, through hole, aperture, and/or other path along longitudinal axis 224 to allow for rod 102 to be disposed therethrough. In one or more embodiments, rotation lock 106 may include a tapered opening to guide rod 102 into the center of rotation lock 106 (and all of the components therein). Rotation lock 106 may include ball bearings 216 and ball pockets 542 that engage with a surface of rod 102, thereby restricting rod rotation 644 of rod 102. Additional details regarding rotation lock 106 may be found in the description of FIGS. 5A-5B and 6A-6B.

Translation control 108 is the controllable linear movement of rod 102 using lock collar 100 and/or linear lock 104. As linear lock 104 may restrict movement of rod 102 with respect to linear lock 104, linear lock 104 may be used to control the movement of rod 102 in that linear direction (i.e., linear lock 104 may be coupled to a motorized track and used to move rod 102).

Rotation control 110 is the controllable rotational movement of rod 102 using lock collar 100 and/or rotation lock 106. As rotation lock 106 may restrict rotation of rod 102 with respect to rotation lock 106, rotation lock 106 may be used to control the rod rotation 644 (i.e., rotation lock 106 may be coupled to a motor and used to rotate rod 102).

As depicted in FIG. 1B, multiple lock collars 100 (lock collar A 100A, lock collar B 100B) may be installed on a single rod 102. In one or more embodiments, lock collars 100 may be installed in opposite directions (“oriented” in a particular direction), where linear lock A 104A and linear lock B 104B limit translation in opposing directions. In turn, as depicted in FIG. 1C, one lock collar 100 (e.g., lock collar A 100A) may be used for translation control 108, while one or both of lock collar A 100A and lock collar B 100B may be used for rotation control 110. In one or more embodiments, where lock collars 100 move closer due to translation control 108, the lock collar 100 which is not used for translation control 108 (e.g., lock collar B 100B) may be shifted along rod 102, thereby separating lock collar A 100A and lock collar B 100B.

Rod bending system 101 is collection of mechanisms and systems for holding, moving, and bending rod 102. In one or more embodiments, rod bending system 101 may include one or more lock collars 100 to control the linear and rotation movement of rod 102, bender 103 for bending rod 102, and information handling system 1001.

Bender 103 is device used to impart curvature or bends to rod 102. In one or more embodiments, bender 103 may incorporate an extended pin, and/or may be configured to apply force against rod 102 to create desired shapes. Bender 103 may be actuated by a mechanical, hydraulic, or pneumatic system, and controlled by information handling system 1001. Bender 103 may be designed to accommodate rods 102 of various diameters and materials, and/or may include features such as adjustable bending angles, radius control, or automated bending sequences. Bender 103 may be constructed from hardened steel, or other suitable materials.

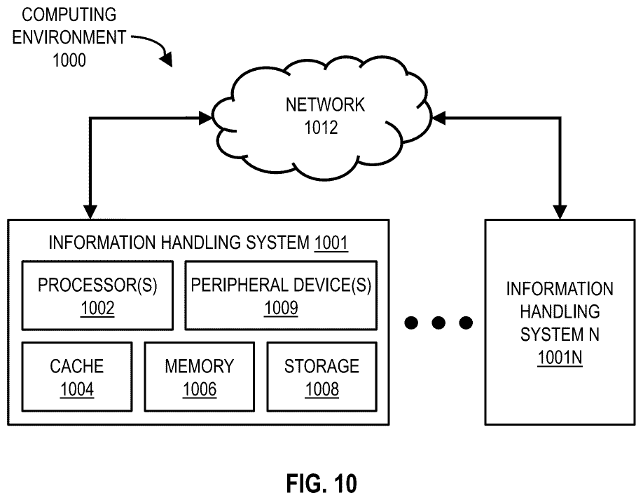

Information handling system 1001 is a hardware computing system which may be operatively connected to one or more components of rod bending system 101 (e.g., lock collar A 100A, lock collar B 100B, bender 103). In one or more embodiments, information handling system 1001 may be used to control the linear position and rotation of rod 102 via lock collars 100 and bend the rods via bender 103. Additional details regarding information handling system 1001 may be found in the description of FIG. 10.

### FIGS. 2A-2B

FIG. 2A is a cutaway diagram showing an exploded view of a linear lock and the components thereof. FIG. 2B is a cutaway diagram showing an assembled view of a linear lock and the components thereof.

Ramp collar 210 is structure in linear lock 104 which includes one or more tapered surfaces 212. In one or more embodiments, ramp collar 210 may be circular in shape (or any shape matching the cross-section of rod 102).

Tapered surface 212 is surface of ramp collar 210 which does not extend parallel to the surface of rod 102 along longitudinal axis 224. In one or more embodiments, tapered surface 212 may take the form of a conical frustum, with the outer surface defined on ramp collar 210 and the interior region unobstructed. Tapered surface 212 may be more proximate to rod 102 on one end and less proximate to rod 102 at the opposite, along longitudinal axis 224.

Slot 214 is cavity (or other negative space) in cage 218 which allows ball bearing 216 to be disposed, at least partially, therein. Slot 214 may generally be defined by a constant width (or diameter) sufficiently large enough to permit ball bearing 216 to protrude, at least partially, therethrough. Slot 214 may include a narrower region (e.g., a “neck”) at one or both ends to prevent ball bearing 216 from fully traversing slot 214 (and becoming misplaced). However, ball bearing 216 may still protrude partially through the opening of the narrower region (e.g., to maintain contact with rod 102 and/or tapered surface 212).

Ball bearing 216 is a spherical structure which is configured to maintain contact with one or more surfaces. As used in linear lock 104, one or more ball bearings 216 may be used to maintain contact with tapered surface 212 and the outer surface of rod 102. Further, ball bearing 216 may rotate (ball rotation 332) and translate (ball translation 334) (e.g., “roll”) along tapered surface 212. Ball bearing 216 may be disposed within slot 214 of cage 218. Further, in one or more embodiments, as ball bearing 216 translates along the tapered surface 212, ball bearing 216 may place a force on cage 218 (via slot 214) to cause movement of cage 218 (cage translation 336). One or more ball bearing 216 may be used in rotation lock 106, with more details found in the description of FIGS. 5A-5B and 6A-6B.

Cage 218 is a structure of linear lock 104 and may be disposed inside of outer frame 222. In one or more embodiments, cage 218 may incorporate one or more slots 214 configured to retain one or more ball bearings 216, respectively. Cage 218 may be designed to undergo cage translation 336 along longitudinal axis 224, caused by forces applied by ball bearings 216. Cage 218 may be constructed from a material with low friction characteristics to enable smooth translation.

Spring 220 is a component configured to place tension between cage 218 and outer frame 222. In one or more embodiments, where no counteracting forces are present, spring 220 causes cage 218 to move towards ramp collar 210. In one or more embodiments, spring 220 may be a compression spring, may be in the form of a helical coil, and may be made constructed from steel (e.g., spring steel). As depicted in FIGS. 2A and 2B, spring 220 may be helically disposed around cage 218 and within the cylindrical contours of outer frame 222. Further, spring 220 may be disposed against a flange, lip, shoulder, or other protrusion of cage 218 and outer frame 222 along longitudinal axis 224.

Outer frame 222 is a structure forming the chassis of linear lock 104. In one or more embodiments, outer frame 222 may establish a form factor for linear lock 104. Outer frame 222 may serve to contain and/or protect internal components of linear lock 104. Outer frame 222 may provide a surface for attachment to other components (e.g., to rotation lock 106, for translation control 108, etc.). Outer frame 222 may include features such as mounting points, alignment guides, and/or structural reinforcements.

Longitudinal axis 224 is a line extending along a length of an object or space. In one or more embodiments, longitudinal axis 224 may be defined as a central line about which rod 102, linear lock 104, and/or rotation lock 106 are arranged. Longitudinal axis 224 can be considered a reference for describing the spatial relationship and alignment of these components. In one or more embodiments, a structural member, such as rod 102, may be considered to extend along longitudinal axis 224. Additionally, locations of linear lock 104 and rotation lock 106 may be described in relation to longitudinal axis 224.

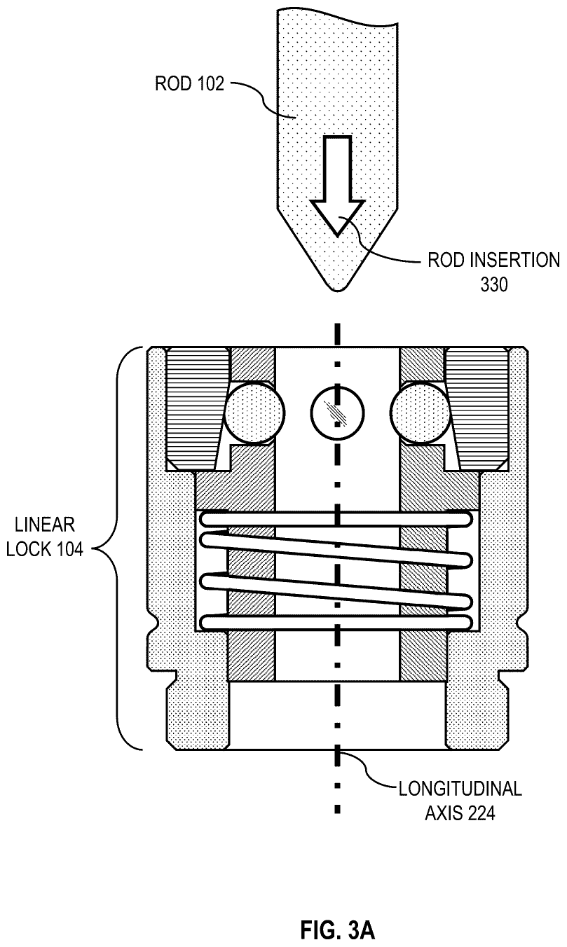

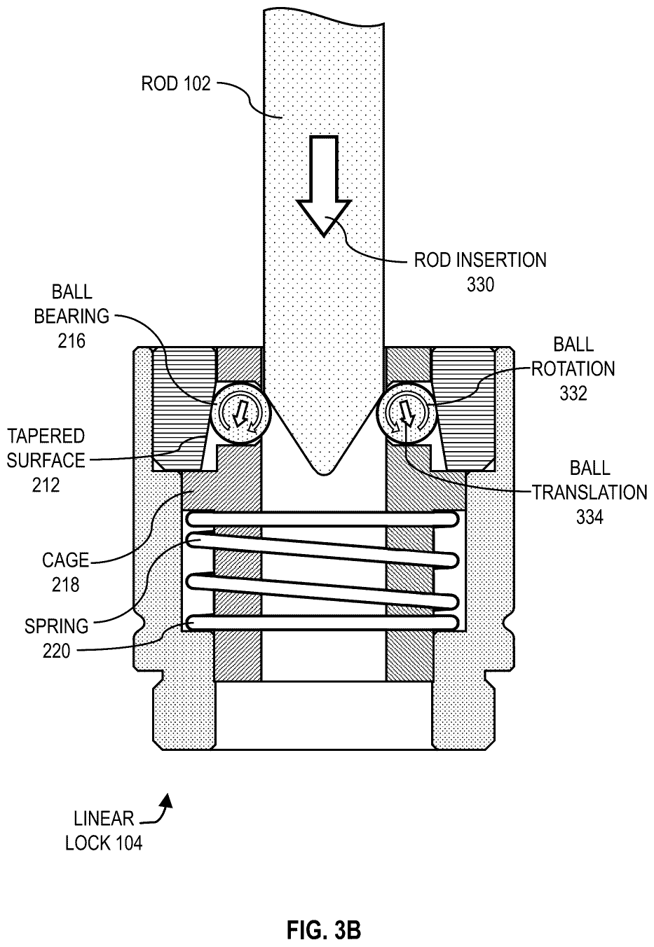

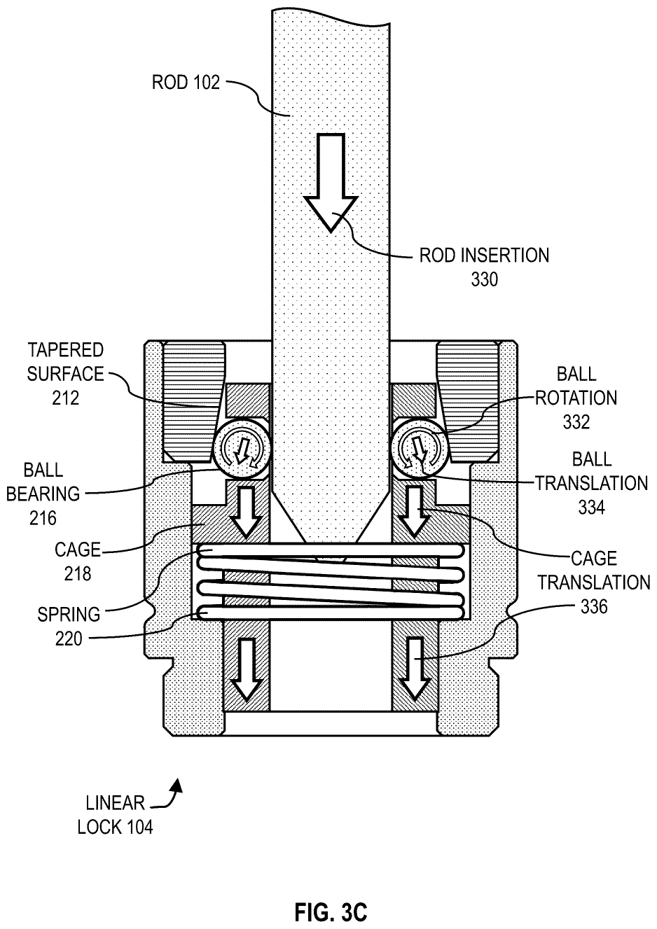

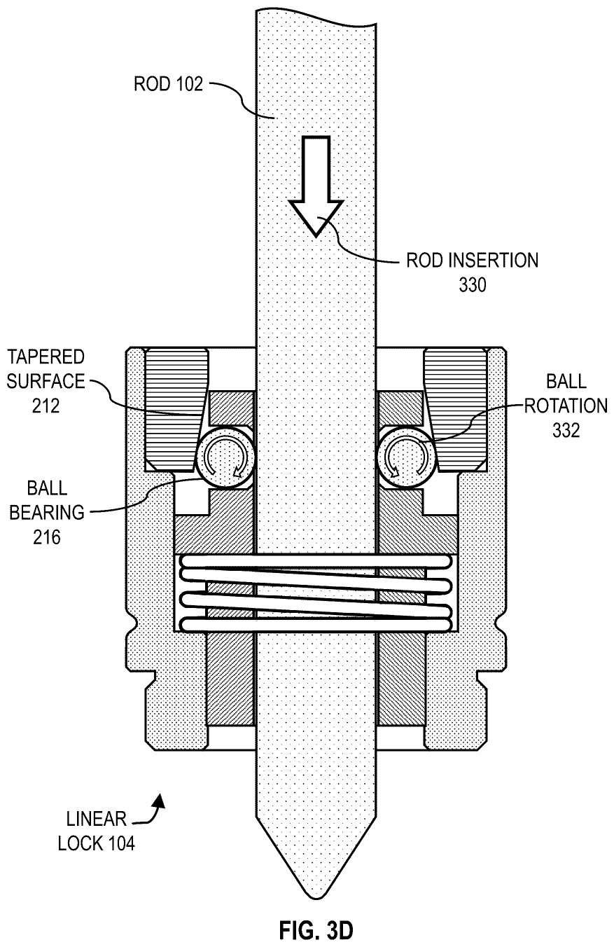

### FIGS. 3A-3D

FIG. 3A is a diagram of a rod approaching a linear lock. FIG. 3B is a diagram of a rod entering a linear lock. FIG. 3C is a diagram of a rod displacing ball bearings and shifting the cage. FIG. 3D is a diagram of a rod fully traversed through a linear lock.

Rod insertion 330 is an act of moving rod 102 into one or more mechanisms and/or structures (e.g., linear lock 104, rotation lock 106, rod bending system 700, etc.). Rod insertion 330 may be performed manually, automatically, and/or through a combination thereof. Rod insertion 330 may be guided by alignment features present on linear lock 104 (e.g., a tapered opening, empty central volume allowing rod 102 to translate therethrough). Rod insertion 330 may result in engagement between rod 102 and internal components of linear lock 104, such as ball bearings 216 and/or cage 218.

Ball rotation 332 is the revolving, circular, and/or spinning movement of ball bearing 216. Ball rotation 332 may occur in response to an applied force, such as the insertion or movement of rod 102. Ball rotation 332 may facilitate a low-friction translation or rotation between components. Ball rotation 332 may be constrained by cage 218, ramp collar 210, and/or rod 102.

Ball translation 334, in linear lock 104, is a linear movement ball bearing 216 along (and guided by the geometry of) tapered surface 212 and/or a leading conical end of rod 102. Ball translation 334 may occur in response to an axial force applied to ball bearing 216 (e.g., by rod insertion 330). In one or more embodiments, ball translation 334 may cause ball bearing 216 to place a force on the side of slot 214 (and cage 218 generally). Such forces, if sufficient enough to overcome counteracting forces created by spring 220 (and other friction), causes linear movement of cage 218 (cage translation 336), compressing spring 220 (in a direction away from ramp collar 210, as depicted).

Cage translation 336 is the linear movement of cage 218 along longitudinal axis 224. Linear movement of cage 218 may be constrained by ramp collar 210, outer frame 222, and/or spring 220. In one or more embodiments, when one or more ball bearings 216 undergo ball translation 334 along tapered surface 212, those ball bearings 216 may place a force on cage 218 (via ball bearing 216 contact with the contours of slot 214) causing cage 218 to undergo cage translation 336. Cage translation 336 may cause spring 220 to compress between outer frame 222 and cage 218. In one or more embodiments, after rod 102 fully traverses linear lock 104 (i.e., a rear end of rod 102 exits linear lock 104 through the opening opposite to ramp collar 210), cage 218 may undergo cage translation 336 in the opposite direction, moving towards ramp collar 210 and allowing spring 220 to expand.

In FIG. 3A, rod 102 undergoes rod insertion 330 and moves towards an opening of linear lock 104.

In FIG. 3B, rod 102, undergoing rod insertion 330, begins to traverse into the inner central path of linear lock 104 (along longitudinal axis 224). The leading pointed edge of rod 102 makes contact with one or more ball bearings 216, causing those ball bearings to undergo ball rotation 332 and ball translation 334.

In FIG. 3C, rod 102, undergoing rod insertion 330, fully displaces ball bearings 216 radially outward along tapered surface 212. Further, due to ball translation 334, ball bearings 216 caused cage 218 to undergo cage translation 336 and compress spring 220.

In FIG. 3D, rod 102, undergoing rod insertion 330, fully traverses through the length of linear lock 104, with the leading pointed edge exposed on the outside of linear lock 104. One or more ball bearings 216 may continue to undergo ball rotation 332 as rod insertion 330 continues. However, ball translation 334 and cage translation 336 do not continue as the geometry of slots 214, cage 218, spring 220, and outer frame 222 may limit further movement.

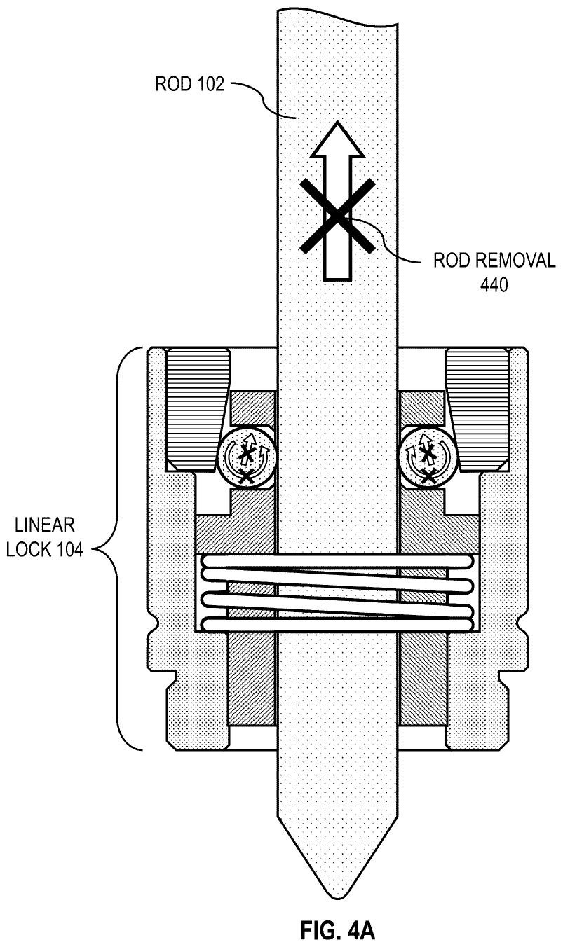

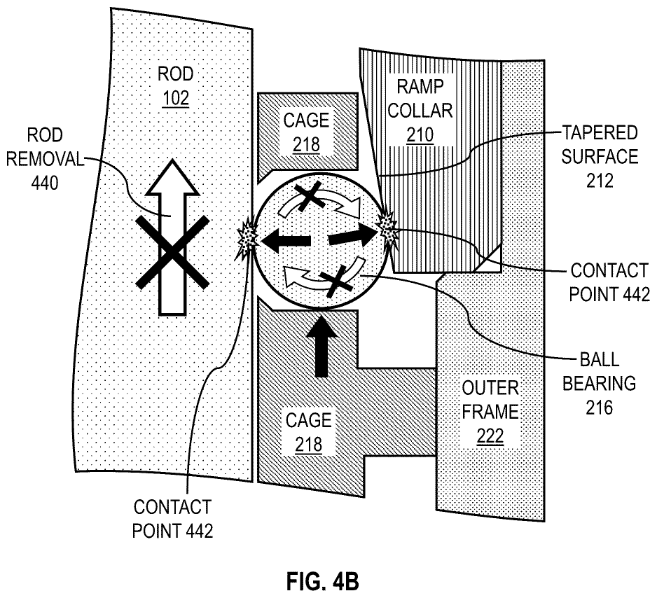

### FIGS. 4A-4B

FIG. 4A is a diagram showing a rod attempting to be removed from a linear lock. FIG. 4B is a diagram showing a closeup of the rod interacting with a ball bearing during rod removal.

Rod removal 440 is an act of moving rod 102 out of one or more mechanisms and/or structures (e.g., linear lock 104, rotation lock 106, rod bending system 700, etc.) in a direction opposite to rod insertion 330. Rod removal 440 may be performed manually, automatically, and/or through a combination thereof. Rod removal 440 may be guided by alignment features present on linear lock 104 (e.g., a tapered opening, empty central volume allowing rod 102 to translate therethrough). In one or more embodiments, linear lock 104 may prevent (or otherwise increase the force required to perform) rod removal 440.

Contact point 442 is a location where the surface of two structure mate, engage, or otherwise touch. As depicted in FIG. 4B, two contact points 442 are shown where ball bearing 216 makes contact with rod 102 and tapered surface 212.

As depicted in FIGS. 4A and 4B, when rod removal 440 is attempted, one or more ball bearings 216 attempt a reverse ball rotation 332 (in the opposite direction as caused by rod insertion 330) due to the friction at contact points 442. In turn, the reverse ball rotation 332 causes ball bearing 216 to (attempt to) undergo a reverse ball translation 334 along tapered surface 212. Additionally, cage 218 places force on ball bearing 216 (via spring 220) to further (attempt to) cause reverse ball translation 334.

However, due to the distance between rod 102 and ramp collar 210, there is not sufficient volume to allow for ball bearing 216 to undergo the reverse ball translation 334. Instead, ball bearing 216 is forced into the narrow wedge created between rod 102 and ramp collar 210. In turn, ball bearing 216 exerts force outward at contact points 442. In one or more embodiments, the normal forces at contact points 442 causes friction (increasing and decreasing directly in a linear relationship with the normal force) between ball bearing 216 and rod 102. In turn, the friction between ball bearing 216 and rod 102 may be sufficient to prevent (or otherwise increase the force required to perform) rod removal 440.

### FIGS. 5A-5B

FIG. 5A is a cutaway diagram showing the components of a rotation lock. FIG. 5B is a cutaway diagram showing the components of a rotation lock with a rod inserted therethrough.

Body 540 is a structure forming the chassis of rotation lock 106. In one or more embodiments, body 540 may establish a form factor for rotation lock 106. Body 540 may serve to contain and/or protect internal components of rotation lock 106. Body 540 may provide a surface for attachment to other components (e.g., to linear lock 104, for rotation control 110, etc.). Body 540 may include features such as mounting points, alignment guides, and/or structural reinforcements.

Ball pocket 542 is cavity (or other negative space) in body 540 which allows ball bearing 216 to be disposed fully or partially therein. Ball pocket 542 may generally be shaped to permit ball bearing 216 to protrude, at least partially, towards the center of rotation lock 106 (i.e., towards longitudinal axis 224) to contact rod 102. In one or more embodiments, without rod 102 disposed in rotation lock 106, ball bearings 216 may move freely in ball pocket 542, but are prevented from exiting ball pocket 542 due to the opening of ball pocket 542 (disposed towards longitudinal axis 224) being narrower than the diameter of ball bearing 216.

In one or more embodiments, when rod 102 disposed in rotation lock 106, ball bearings 216 are pushed radially outward further into their respective ball pockets 542. The geometry of ball pocket 542 allows for ball bearings 216 to rest with no (or minimal) compressive forces between ball pocket 542 and rod 102. However, the surface of ball pocket 542 (opposite from rod 102 and/or longitudinal axis 224) tapers inwards towards the center of rotation lock 106 and does not leave sufficient space for ball bearing 216 to move laterally. Accordingly, on both lateral sides of ball bearing 216, a volume exists in which ball bearing 216 may become wedged if caused to move in those lateral directions.

### FIGS. 6A-6B

FIG. 6A is a diagram showing a rod attempting to rotate in a rotation lock. FIG. 6B is a diagram showing a closeup of the rod interacting with a ball bearing during rod rotation.

Rod rotation 644 is the revolving, circular, and/or spinning movement of rod 102. Rod rotation 644 may occur in response to an applied torque on rod 102. Rod rotation 644 may cause ball rotation 332 on one or more ball bearings 216 in contact with rod 102. In one or more embodiments, rotation lock 106 may prevent (or otherwise increase the torque required to perform) rod rotation 644.

As depicted in FIGS. 6A and 6B, when rod rotation 644 is attempted, one or more ball bearings 216 attempt ball rotation 332 in one or more ball pockets 542 due to the friction at contact points 442. In turn, the ball rotation 332 causes ball bearing 216 to undergo some ball translation 334 along the surface of ball pocket 542 (opposite from rod 102). However, due to the distance between rod 102 and the surface of ball pocket 542, there is not sufficient volume to allow for ball bearing 216 to undergo further ball translation 334. Instead, ball bearing 216 is forced into the wedge between rod 102 and the opposite surface of ball pocket 542. Consequently, ball bearing 216 exerts force at contact points 442. In one or more embodiments, the normal forces at contact points 442 causes friction (increasing and decreasing directly in a linear relationship with the normal force) between ball bearing 216 and rod 102. That friction may be sufficient to prevent (or otherwise increase the torque required to perform) rod rotation 644.

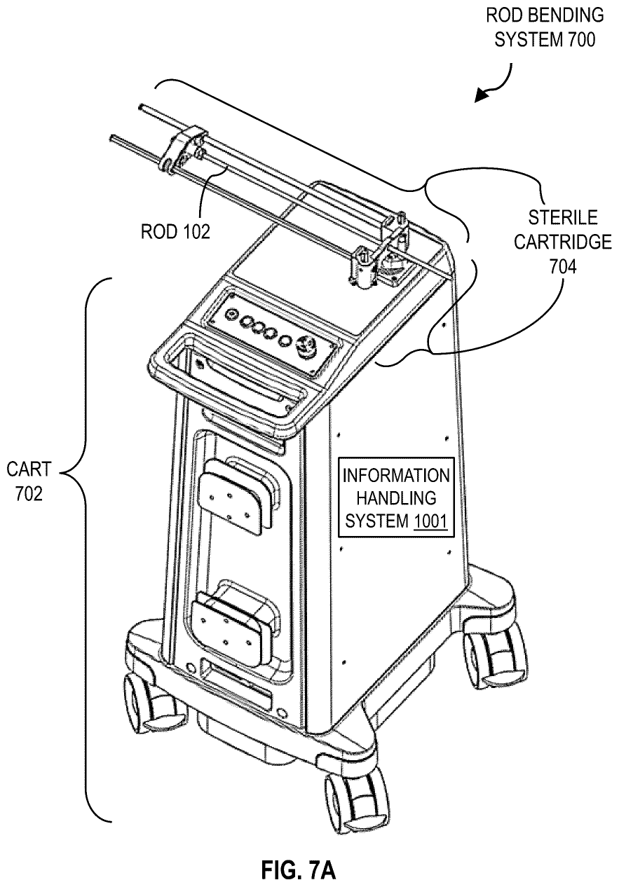

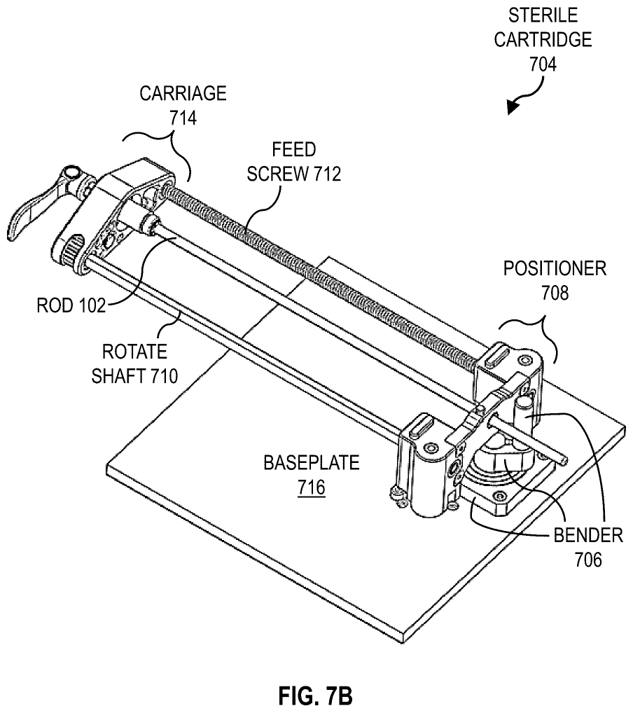

### FIGS. 7A-7B

FIG. 7A is a diagram showing a rod bending system and the components thereof. FIG. 7B is a diagram showing a sterile cartridge and the components thereof.

Rod bending system 700 is collection of mechanisms and systems for holding, moving, and bending rod 102. In one or more embodiments, rod bending system 700 may include cart 702, information handling system 1001, and sterile cartridge 704. Each of these components is described below.

Information handling system 1001 is a hardware computing system which may be operatively connected to one or more components of sterile cartridge 704 (e.g., bender 706 and/or positioner 708). In one or more embodiments, information handling system 1001 may be used to control bender 706 and/or positioner 708 to move (translate and rotate) and modify (bend) rod 102. Additional details regarding information handling system 1001 may be found in the description of FIG. 10.

Cart 702 is a mobile platform designed to support sterile cartridge 704. In one or more embodiments, cart 702 may be separated from sterile cartridge 704 by a sterile drape, and/or may be constructed from materials suitable for use in a sterile environment. Cart 702 may incorporate information handling system 1001, power sources, motors, engines, and gear mechanisms for controlling positioner 708 and bender 706. Cart 702 may be equipped with wheels or casters to facilitate movement, and/or may include locking mechanisms to secure its position. Cart 702 may be designed to provide a stable and reliable support structure for sterile cartridge 704, and/or may include features such as adjustable height, integrated cable management, or storage compartments.

Sterile cartridge 704 is a combination of mechanisms and devices which may be assembled on top of cart 702. Sterile cartridge 704 may be separated from cart 702 by a sterile drape (not shown) to maintain a sterile barrier between the components in cart 702 and the components of sterile cartridge 704. Sterile cartridge 704 may include baseplate 716, bender 706, positioner 708, carriage 714, rotate shaft 710, and feed screw 712.

Bender 706 is a device used to impart curvature or bends to rod 102. In one or more embodiments, bender 706 may incorporate an extended pin, and/or may be configured to apply force against rod 102 to create desired shapes. Bender 706 may be disposed proximate to the opening of mandrel 824. Bender 706 may be actuated by a mechanical, hydraulic, or pneumatic system (e.g., within cart 702) and controlled by information handling system 1001. Bender 706 may be designed to accommodate rods 102 of various diameters and materials, and/or may include features such as adjustable bending angles, radius control, or automated bending sequences. Bender 706 may be constructed from hardened steel, or other suitable materials.

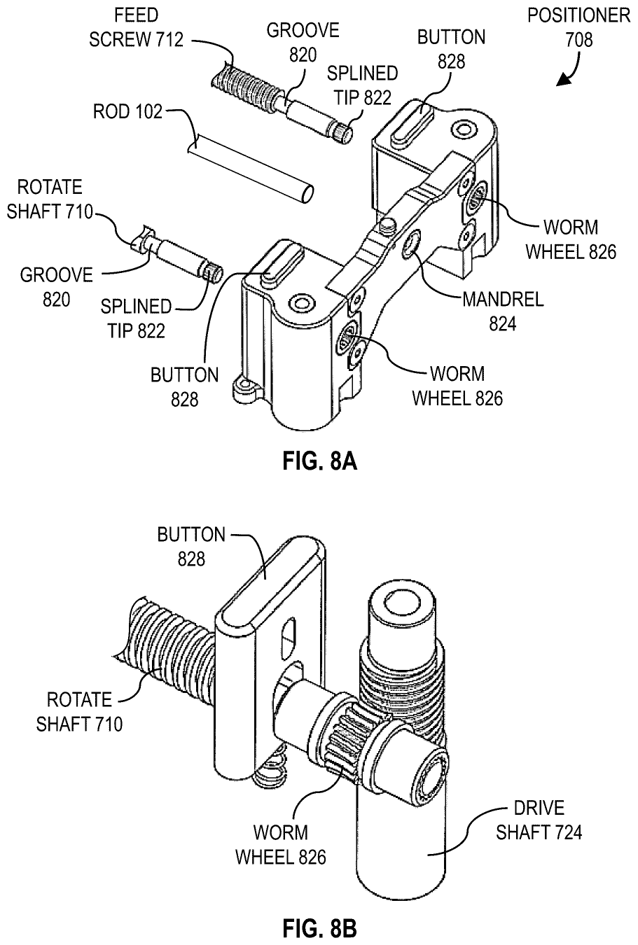

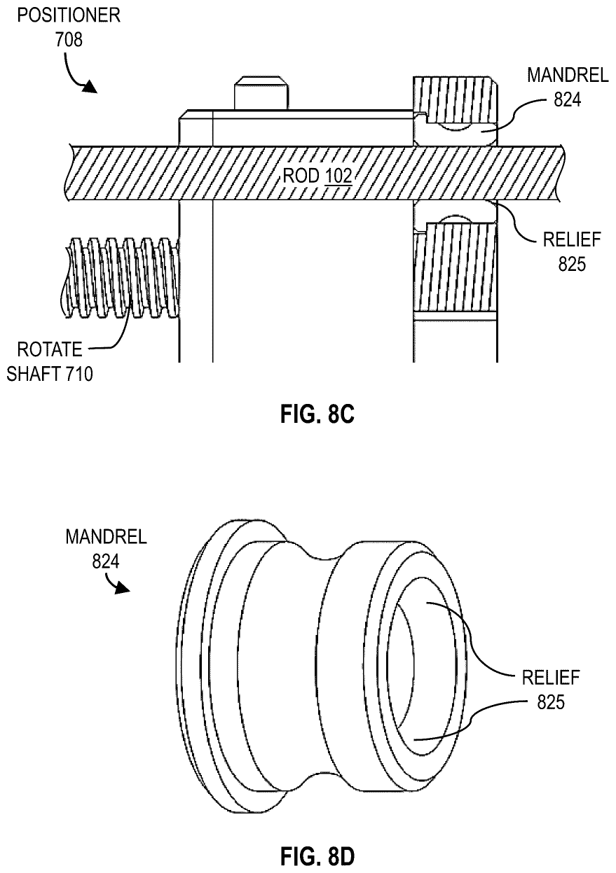

Positioner 708 is a mechanical assembly configured to control the linear and angular movements of rod 102. In one or more embodiments, positioner 708 may house two drive shafts 724, one for rotate shaft 710 and one for feed screw 712. Positioner 708 may be designed to independently control the rotation of rotate shaft 710 and the translation of feed screw 712. Positioner 708 may be controlled by information handling system 1001 to precisely position rod 102 with respect to bender 706. Positioner 708 may incorporate features such as bearings, gears, and/or other mechanical components to facilitate smooth and accurate movement. Positioner 708 may facilitate rod insertion 330 and rod rotation 644. Additional details regarding positioner 708 may be found in the description of FIGS. 8A-8D.

Rotate shaft 710 is a mechanical component which transmits rotational motion. In one or more embodiments, rotate shaft 710 may be coupled to a power source, such as a motor or engine (attached to drive shaft 724 in positioner 708), to initiate rotation. Rotate shaft 710 may be supported by bearings or other support structures to minimize friction and maintain alignment, and/or may be designed with varying diameters, lengths, and/or cross-sectional shapes to accommodate different torque and speed requirements. Rotate shaft 710 may incorporate features such as keyways, splines, or threaded ends to facilitate the attachment of gears, pulleys, or other rotating components. In one or more embodiments, rotate shaft 710 is coupled to rod 102 via carriage 714 to cause rotation of rod 102.

Feed screw 712 is a helical-threaded shaft used to convey carriage 714 along an axis (towards positioner 708). In one or more embodiments, feed screw 712 may be rotated by a motor or engine (attached to drive shaft 724 in positioner 708) or other mechanical means, and/or may engage with a corresponding nut or threaded component to translate rotational motion into linear motion (e.g., via worm wheel 826). Feed screw 712 may be designed with varying thread pitches, diameters, and/or lengths to control the rate and precision of material or component movement. As feed screw 712 may be used to move carriage 714 towards positioner 708, rod 102 is also translated towards positioner 708.

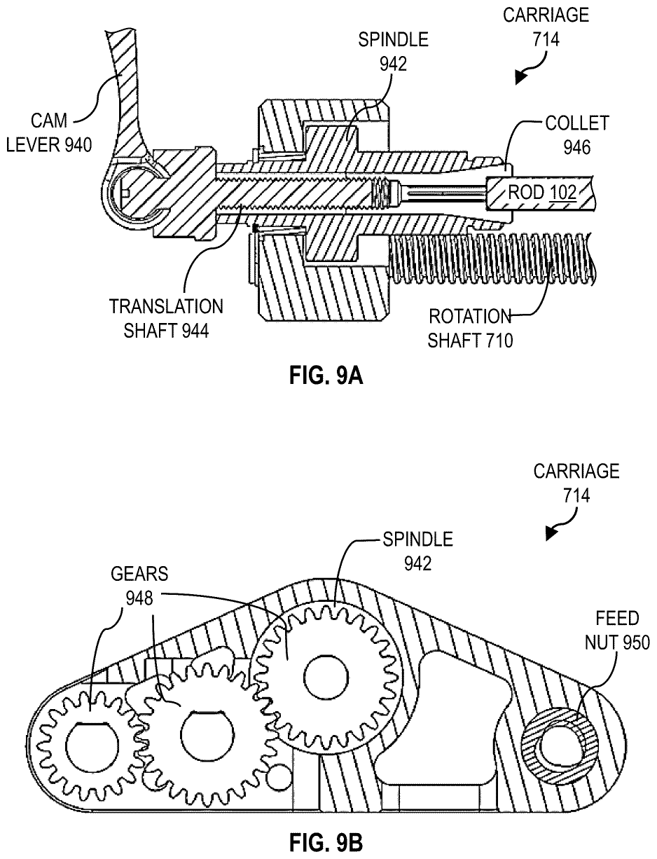

Carriage 714 is a mechanism which may be used to facilitate the translation and rotation of rod 102. In one or more embodiments, carriage 714 is connected to rotate shaft 710 and feed screw 712, which may control the rotation of rod 102 and linear movement of carriage 714, respectively. Carriage 714, via spindle 942 and collet 946, may grip and remain fixed with respect to one end of rod 102. Additional details regarding carriage 714 may be found in the description of FIGS. 9A-9B.

### FIGS. 8A-8D

FIG. 8A is a diagram showing an exploded view of a positioner and the components thereof. FIG. 8B is a diagram showing a button and surrounding components of a positioner. FIG. 8C is a cutaway diagram showing a positioner and the components thereof. FIG. 8D is a diagram showing a mandrel.

Groove 820 is narrow region on rotate shaft 710 and/or feed screw 712. Grooves 820 may have a smaller diameter than surrounding portions of rotate shaft 710 and/or feed screw 712. Grooves 820 may be used to lock rotate shaft 710 and/or feed screw 712 into positioner 708 by engaging the thinner circumference and limiting linear movement. In one or more embodiments, one or more grooves 820 engage with one or more buttons 828 in positioner 708, respectively.

Splined tip 822 is an end of rotate shaft 710 and/or feed screw 712 disposed in positioner 708. In one or more embodiments, splined tips 822 are used to rotate shaft 710 and/or feed screw 712 via engagement with worm wheel 826 (and drive shaft 724).

Mandrel 824 is a support component configured to hold rod 102 as it passes through positioner 708. Further, mandrel 824 may be used to maintain the linear and angular position of rod 102 immediately prior to bending by bender 706. In one or more embodiments, mandrel 824 may be a cylindrical or shaped ring. Mandrel 824 may be designed to have a smooth central surface to minimize friction with rod 102, and/or may incorporate features such as grooves, slots, or other locating elements to ensure accurate positioning of rod 102. Mandrel 824 may be secured within positioner 708 by fasteners or other suitable means, and/or may be configured to withstand bending forces applied to rod 102 during the bending process. Mandrel 824 may be available in multiple sizes to accommodate respective sizes of rod 102.

Relief 825 is a feature at one or both openings of mandrel 824 configured to reduce stress concentration. In one or more embodiments, relief 825 may be a curved or rounded surface, and/or may be formed by machining, grinding, or other suitable techniques. Relief 825 may be radiused, toroidal, or of other suitable geometry configured to provide a smooth transition between the opening of mandrel 824 and the outer surface of rod 102. Relief 825 may prevent or minimize damage to rod 102 during rod insertion 330, rod removal 440, and bending by bender 706.

Worm wheel 826 is a gear component, fixed in positioner 708, and adapted to accept splined tip 822 of rotate shaft 710 and/or feed screw 712. Positioner 708 may include multiple worm wheels 826 for mating with corresponding splined tips 822. In one or more embodiments, worm wheel 826 may feature teeth that engage with a drive shaft 724, allowing drive shaft 724 to cause rotation of worm wheel 826 and splined tip 822. Worm wheel 826 may be designed with a specific tooth profile to ensure smooth and efficient engagement with drive shaft 724. Worm wheel 826 may be supported by bearings or other suitable means to minimize friction and maintain alignment, and/or may be used to transmit rotational motion at a reduced speed and/or increased torque.

Button 828 is a manually actuated, spring-loaded component installed within positioner 708. In one or more embodiments, button 828 may incorporate a cutout to engage with groove 820 of rotate shaft 710 and/or feed screw 712. Button 828 may be configured to maintain a specific position of rotate shaft 710 and/or feed screw 712. Button 828 may be designed to be manually depressed, thereby disengaging the cutout from groove 820, and/or may be returned to its engaged position by a spring or other biasing mechanism. Button 828 may be used to provide a detent or locking function, and/or may be configured to prevent unintended rotation or axial movement of rotate shaft 710 and/or feed screw 712.

### FIGS. 9A-9B

FIG. 9A is a side cutaway diagram showing a carriage and the components thereof. FIG. 9B is a front cutaway diagram showing a carriage and the components thereof.

Cam lever 940 is a mechanical component used to actuate translation shaft 944, thereby tightening (or loosening) collet 946 around rod 102. In one or more embodiments, cam lever 940 may incorporate an eccentric cam profile, and/or may pivotally mount to carriage 714. Cam lever 940 may be equipped with a handle for manual control, and/or may be configured to provide a mechanical advantage for applying force to translation shaft 944. Cam lever 940 may include features such as a locking mechanism, detents, or adjustable stops. Cam lever 940 may translate rotational motion to linear motion, and/or may be designed to provide a specific clamping force to collet 946.

Spindle 942 is a rotating component designed to hold collet 946, and/or to impart rotational motion to collet 946 when driven by rotate shaft 710. In one or more embodiments, spindle 942 may be rigidly attached to gear 948 (gear 948 centered around collet 946). Spindle 942 may be designed with a central bore or other feature to accommodate collet 946. Spindle 942 may incorporate features such as a keyway, spline, or threaded connection to secure gear 948, and/or may be configured to transmit torque from rotate shaft 710 to collet 946. Spindle 942 may be designed to maintain concentricity, alignment during rotation, and/or precise rotational control.

Translation shaft 944 is a mechanical component that converts rotational motion of cam lever 940 into linear motion for tightening collet 946. In one or more embodiments, translation shaft 944 may be threaded, and/or may engage with a corresponding nut or threaded component within collet 946. Translation shaft 944 may be configured to transmit force from cam lever 940 to collet 946. Translation shaft 944 may incorporate features such as a keyway, spline, or flat surface to prevent rotation, and/or may be designed with specific thread pitches and lengths to achieve desired clamping forces.

Collet 946 is a holding device configured to secure rod 102. In one or more embodiments, collet 946 may incorporate a series of tapered segments or jaws, and/or may be actuated by translation shaft 944 to clamp rod 102. Collet 946 may be designed to accommodate rods 102 of various diameters and materials, and/or may provide concentric clamping to ensure accurate positioning of rod 102. Collet 946 may be constructed from hardened steel, or other suitable materials, and/or may include features such as internal threads, slots, or keyways to facilitate attachment to a machine or other holding fixture. Collet 946 may be available in multiple sizes to accommodate respective sizes of rod 102.

Gears 948 are a series of toothed wheels (i.e., cogs) that form a gear train, and are used to transmit rotational motion from rotate shaft 710 to rod 102. In one or more embodiments, gears 948 may be of various sizes and tooth counts, and/or may be arranged to achieve a desired gear ratio. Gears 948 may be housed within a gear case or other protective enclosure (e.g., case of carriage 714). Gears 948 may be configured to provide a specific speed or torque output to rod 102.

Feed nut 950 is a component fixed within carriage 714 that engages with feed screw 712 to facilitate linear movement of carriage 714. In one or more embodiments, feed nut 950 may be constructed from a wear-resistant material, and/or may be designed with internal threads that match the thread profile of feed screw 712. Feed nut 950 may be configured to translate the rotational motion of feed screw 712 into linear motion of carriage 714. Feed nut 950 may be secured to carriage 714 by fasteners or other suitable means, and/or may be designed to provide a specific lead or pitch to control the rate of carriage 714 movement.

### FIG. 10

FIG. 10 is a diagram of an example computing environment. Computing environment 1000 may include one or more information handling systems 1001 connected via network 1012. Each of these components is described below.

Information handling system 1001 is a hardware computing device which may be utilized to perform various steps, methods, and techniques disclosed herein (e.g., via the execution of software). In any embodiment, information handling system 1001 may include one or more processors 1002, cache 1004, memory 1006, storage 1008, and/or one or more peripheral devices 1009. Any two or more of these components may be operatively connected via a system bus (not shown) which provides a means for transferring data between those components. Although each component is depicted and disclosed as individual functional components, these individual components may be combined (or divided) into any combination or configuration of components.

A system bus is a system of hardware connections (e.g., sockets, ports, wiring, conductive tracings on a printed circuit board (PCB), etc.) used for sending (and receiving) data to (and from) each of the components connected thereto. In any embodiment, a system bus allows for communication via an interface and protocol (e.g., inter-integrated circuit (I2C), peripheral component interconnect (express) (PCI(e)) fabric, etc.) which may be commonly recognized by the components utilizing the system bus. In any embodiment, a basic input/output system (BIOS) may be configured to transfer information between the components using the system bus (e.g., during initialization of information handling system 1001).

In any embodiment, information handling system 1001 may additionally include internal physical interfaces (e.g., serial advanced technology attachment (SATA) ports, peripheral component interconnect (PCI) ports, PCI express (PCIe) ports, next generation form factor (NGFF) ports, M.2 ports, etc.) and/or external physical interfaces (e.g., universal serial bus (USB) ports, recommended standard (RS) serial ports, audio/visual ports, etc.). Internal physical interfaces and external physical interfaces may facilitate the operative connection to one or more peripheral devices 1009.

Non-limiting examples of information handling system 1001 include a general purpose computer (e.g., a personal computer, desktop, laptop, tablet, smart phone, etc.), a network device (e.g., switch, router, multi-layer switch, etc.), a server (e.g., a blade-server in a blade-server chassis, a rack server in a rack, etc.), a controller (e.g., a programmable logic controller (PLC)), and/or any other type of computing device with the aforementioned capabilities. Further, information handling system 1001 may be operatively connected to another information handling system 1001 via network 1012 in a distributed computing environment. As used herein, a “computing device” may be equivalent to an information handling system.

Processor 1002 is a hardware device which may take the form of an integrated circuit configured to process computer-executable instructions (e.g., software). Processor 1002 may execute (e.g., read and process) computer-executable instructions stored in cache 1004, memory 1006, and/or storage 1008. Processor 1002 may be a self-contained computing system, including a system bus, memory, cache, and/or any other components of a computing device. Processor 1002 may include multiple processors, such as a system having multiple, physically separate processors in different sockets, or a system having multiple processor cores on a single physical chip. A multi-core processor may be symmetric or asymmetric. Multiple processors 1002, and/or processor cores thereof, may share resources (e.g., cache 1004, memory 1006) or may operate using independent resources.

Non-limiting examples of processor 1002 include general-purpose processor (e.g., a central processing unit (CPU)), an application specific integrated circuit (ASIC), a programmable gate array (PGA), a field programmable gate array (FPGA), a digital signal processor (DSP), and any digital or analog circuit configured to perform operations based on input data (e.g., execute program instructions).

Cache 1004 is one or more hardware devices capable of storing digital information (e.g., data) in a non-transitory medium. Cache 1004 expressly excludes transitory media (e.g., transitory waves, energy, carrier signals, electromagnetic waves, signals per se, etc.). Cache 1004 may be considered “high-speed”, having comparatively faster read/write access than memory 1006 and storage 1008, and therefore utilized by processor 1002 to process data more quickly than data stored in memory 1006 or storage 1008. Accordingly, processor 1002 may copy needed data to cache 1004 (from memory 1006 and/or storage 1008) for comparatively speedier access when processing that data. In any embodiment, cache 1004 may be included in processor 1002 (e.g., as a subcomponent). In any embodiment, cache 1004 may be physically independent, but operatively connected to processor 1002.

Memory 1006 is one or more hardware devices capable of storing digital information (e.g., data) in a non-transitory medium. Memory 1006 expressly excludes transitory media (e.g., transitory waves, energy, carrier signals, electromagnetic waves, signals per se, etc.). In any embodiment, when accessing memory 1006, software (executed via processor 1002) may be capable of reading and writing data at the smallest units of data normally accessible (e.g., “bytes”). Specifically, memory 1006 may include a unique physical address for each byte stored thereon, thereby enabling the ability to access and manipulate (read and write) data by directing commands to a specific physical address associated with a byte of data (i.e., “random access”). Non-limiting examples of memory 1006 devices include flash memory, random access memory (RAM), dynamic RAM (DRAM), static RAM (SRAM), resistive RAM (ReRAM), read-only memory (ROM), and electrically erasable programmable ROM (EEPROM). In any embodiment, memory 1006 devices may be volatile or non-volatile.

Storage 1008 is one or more hardware devices capable of storing digital information (e.g., data) in a non-transitory medium. Storage 1008 expressly excludes transitory media (e.g., transitory waves, energy, carrier signals, electromagnetic waves, signals per se, etc.). In any embodiment, the smallest unit of data readable from storage 1008 may be a “block” (instead of a “byte”). Prior to reading and/or manipulating the data on storage 1008, one or more blocks may be copied to an intermediary storage medium (e.g., cache 1004, memory 1006) where the data may then be accessed in “bytes” (e.g., via random access). In any embodiment, data on storage 1008 may be accessed in “bytes” (like memory 1006). Non-limiting examples of storage 1008 include integrated circuit storage devices (e.g., a solid-state drive (SSD), Non-Volatile Memory Express (NVMe), flash memory, etc.), magnetic storage devices (e.g., a hard disk drive (HDD), floppy disk, magnetic tape, diskette, cassettes, etc.), optical media (e.g., a compact disc (CD), digital versatile disc (DVD), etc.), and printed media (e.g., barcode, quick response (QR) code, punch card, etc.).

As used herein, “non-transitory computer readable medium” is cache 1004, memory 1006, storage 1008, and/or any other hardware device capable of non-transitorily storing and/or carrying data.

Peripheral device 1009 is a hardware device configured to send (and/or receive) data to (and/or from) information handling system 1001 via one or more internal and/or external physical interfaces. Any peripheral device 1009 may be categorized as one or more “types” of computing devices (e.g., an “input” device, “output” device, “communication” device, etc.). However, such categories are not comprehensive and are not mutually exclusive. Such categories are listed herein strictly to provide understandable groupings of the potential types of peripheral devices 1009. As such, peripheral device 1009 may be an input device, an output device, a communication device, and/or any other optional computing component.

An input device is a hardware device which receives data into information handling system 1001. In any embodiment, an input device may be a human interface device which facilitates user interaction by collecting data based on user inputs (e.g., a mouse, keyboard, camera, microphone, touchpad, touchscreen, fingerprint reader, joystick, gamepad, etc.). In any embodiment, an input device may collect data based on raw inputs, regardless of human interaction (e.g., any sensor, logging tool, audio/video capture card, etc.). In any embodiment, an input device may be a reader for accessing data on a non-transitory computer readable medium (e.g., a CD drive, floppy disk drive, tape drive, scanner, etc.).

An output device is a hardware device which sends data from information handling system 1001. In any embodiment, an output device may be a human interface device which facilitates providing data to a user (e.g., a visual display monitor, speakers, printer, status light, haptic feedback device, etc.). In any embodiment, an output device may be a writer for facilitating storage of data on a non-transitory computer readable medium (e.g., a CD drive, floppy disk drive, magnetic tape drive, printer, etc.).

A communication device is a hardware device capable of sending and/or receiving data with one or more other communication devices (e.g., connected to another information handling system 1001 via network 1012). A communication device may communicate via any suitable form of wired interface (e.g., Ethernet, fiber optic, serial communication etc.) and/or wireless interface (e.g., Wi-Fi® (Institute of Electrical and Electronics Engineers (IEEE) 802.11), Bluetooth® (IEEE 802.15.1), etc.) and utilize one or more protocols for the transmission and receipt of data (e.g., transmission control protocol (TCP), user datagram protocol (UDP), internet protocol (IP), remote direct memory access (RDMA), etc.). Non-limiting examples of a communication device include a network interface card (NIC), a modem, an Ethernet card/adapter, and a Wi-Fi® card/adapter.

An optional computing component is any hardware device which operatively connects to information handling system 1001 and extends the capabilities of information handling system 1001. Non-limiting examples of an optional computing components include a graphics processing unit (GPU), a data processing unit (DPU), and a docking station.

As used herein, “software” (e.g., “code”, “algorithm”, “application”, “routine”) is data in the form of computer-executable instructions. Processor 1002 may execute (e.g., read and process) software to perform one or more functions. Non-limiting examples of functions may include reading existing data, modifying existing data, generating new data, and using any capability of information handling system 1001 (e.g., reading existing data from memory 1006, generating new data from the existing data, sending the generated data to a GPU to be displayed on a monitor). Although software physically persists in cache 1004, memory 1006, and/or storage 1008, one or more software instances may be depicted, in the figures, as an external component of any information handling system 1001 which interacts with one or more information handling systems 1001.

Network 1012 is a collection of connected information handling systems (e.g., 1001, 1001N) which allows for the exchange of data and/or the sharing of computing resources therebetween. Non-limiting examples of network 1012 include a local area network (LAN), a wide area network (WAN) (e.g., the Internet), a mobile network, any combination thereof, and any other type of network which allows for the communication of data and sharing of resources among computing devices operatively connected thereto. A person of ordinary skill in the relevant art, having the benefit of this detailed description, would appreciate that a network is a collection of operatively connected computing devices which enables communication between those computing devices.

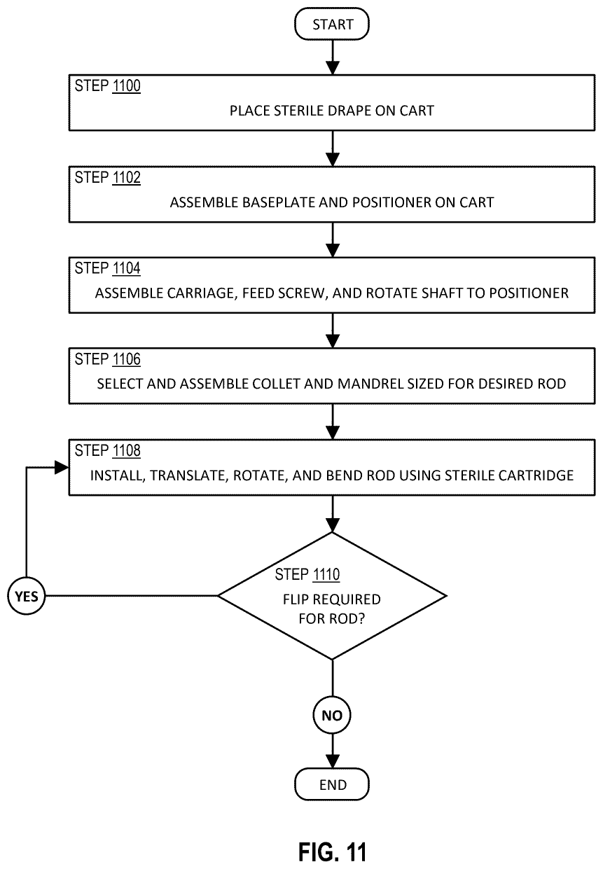

### FIG. 11

FIG. 11 is a flowchart of a method for assembling a rod bending system and bending a rod. All or a portion of the method shown may be performed by one or more components of rod bending system (see description in FIGS. 7A and 7B) or a user thereof. While the various steps in this flowchart are presented and described sequentially, a person of ordinary skill in the relevant art (having the benefit of this detailed description) would appreciate that some or all steps may be executed in different orders, combined, or omitted, and some or all steps may be executed in parallel.

In Step 1100, a sterile drape (not shown) is placed over and/or around cart 702, without sterile cartridge 704 installed on cart 702. A sterile drape may be used to maintain a sterile barrier between cart 702 (which may not be sterile) with the medical environment in which a procedure is being performed (in which a sterile environment is attempted to be maintained).

In Step 1102, baseplate 716 and positioner 708 are installed on cart 702. Sterile drape may include one or more holes to allow for the data communication and mechanical control of sterile cartridge 704.

In Step 1104, carriage 714, feed screw 712, and rotate shaft 710 are assembled to positioner 708 (and baseplate 716). In one or more embodiments, these components, and all components of sterile cartridge 704 are kept sterile.

In Step 1106, collet 946 and mandrel 824 are selected based on the size of rod 102. That is, in one or more embodiments, rod 102 comes in varying sizes (with varying diameters) and thus different collets 946 and mandrel 824 are available for those different diameters. In turn, the appropriate collet 946 and mandrel 824 are selected for the rod 102.

In Step 1108, rod 102 is installed in collet 946 and mandrel 824. Information handling system 1001 is then used to control positioner 708 to translate and/or rotate rod 102 as bender 706 bends rod 102 to a desired shape.

In Step 1110, a determination is made as to whether rod 102 needs to be flipped around and installed in the reverse direction. In one or more embodiments, rod 102 may require bends proximate to both ends of rod 102 and/or rod 102 may have varying diameters through its length (and be unable to traverse mandrel 824). In such instances, it may be required for rod 102 to be removed and reinstalled in carriage 714, at the opposite end, to allow bender 706 to perform the required bends on the opposite side. If a determination is made that a flip is required (Step 1110—YES), the method returns to Step 1108. Alternatively, if a determination is made that a flip is not required (Step 1110—NO), the method may end.

### General Notes

As it is impracticable to disclose every conceivable embodiment of the technology described herein, the figures, examples, and description provided herein disclose only a limited number of potential embodiments. A person of ordinary skill in the relevant art would appreciate that any number of potential variations or modifications may be made to the explicitly disclosed embodiments, and that such alternative embodiments remain within the scope of the broader technology. Accordingly, the scope should be limited only by the attached claims. Further, the compositions and methods are described in terms of “comprising,” “containing,” or “including” various components or steps, the compositions and methods may also “consist essentially of” or “consist of” the various components and steps. Certain technical details, known to those of ordinary skill in the relevant art, may be omitted for brevity and to avoid cluttering the description of the novel aspects.

For further brevity, descriptions of similarly named components may be omitted if a description of that similarly named component exists elsewhere in the application. Accordingly, any component described with respect to a specific figure may be equivalent to one or more similarly named components shown or described in any other figure, and each component incorporates the description of every similarly named component provided in the application (unless explicitly noted otherwise). A description of any component is to be interpreted as an optional embodiment—which may be implemented in addition to, in conjunction with, or in place of an embodiment of a similarly-named component described for any other figure.

### Lexicographical Notes

As used herein, adjective ordinal numbers (e.g., first, second, third, etc.) are used to distinguish between elements and do not create any ordering of the elements. As an example, a “first element” is distinct from a “second element”, but the “first element” may come after (or before) the “second element” in an ordering of elements. Accordingly, an order of elements exists only if ordered terminology is expressly provided (e.g., “before”, “between”, “after”, etc.) or a type of “order” is expressly provided (e.g., “chronological”, “alphabetical”, “by size”, etc.). Further, use of ordinal numbers does not preclude the existence of other elements. As an example, a “table with a first leg and a second leg” is any table with two or more legs (e.g., two legs, five legs, thirteen legs, etc.). A maximum quantity of elements exists only if express language is used to limit the upper bound (e.g., “two or fewer”, “exactly five”, “nine to twenty”, etc.). Similarly, singular use of an ordinal number does not imply the existence of another element. As an example, a “first threshold” may be the only threshold and therefore does not necessitate the existence of a “second threshold”.

As used herein, the term “operative connection” (or “operatively connected”) means the direct or indirect connection between devices which allows for the transmission of data. For example, the phrase ‘operatively connected’ may refer to a direct connection (e.g., a direct wired or wireless connection between devices) or an indirect connection (e.g., multiple wired and/or wireless connections between any number of other devices connecting the operatively connected devices).

As used herein, indefinite articles “a” and “an” mean “one or more”. That is, the explicit recitation of “an” element does not preclude the existence of a second element, a third element, etc. Further, definite articles (e.g., “the”, “said”) mean “any one of” (the “one or more” elements) when referring to previously introduced elements. As an example, there may be “a processor”, where such a recitation does not preclude the existence of any number of other processors. Further, “the processor receives data, and the processor processes data” means “any one of the one or more processors receives data” and “any one of the one or more processors processes data”. It is not required that the same processor both (i) receive data and (ii) process data. Rather, each of the steps (“receive” and “process”) may be performed by different processors.

What is claimed is:

1. A rod bending system, comprising:
a first lock collar, comprising:
a first linear lock; and
a first rotation lock;
a second lock collar, comprising:
a second linear lock; and
a bender configured to bend a rod.

2. The rod bending system of claim 1, wherein the first linear lock comprises:
a ramp collar with a tapered surface;
a cage with a plurality of slots;
a plurality of ball bearings disposed in the plurality of slots, respectively, and disposed against the tapered surface;
an outer frame disposed around the cage; and
a spring disposed between the outer frame and the cage and configured to move the cage towards the ramp collar.

3. The rod bending system of claim 2, wherein during a rod insertion of the rod, the plurality of ball bearings undergo ball translation along the tapered surface.

4. The rod bending system of claim 3, wherein the cage undergoes cage translation away from the tapered surface due to the ball translation.

5. The rod bending system of claim 4, wherein the plurality of ball bearings prevent a rod removal of the rod from the first linear lock.

6. The rod bending system of claim 1, wherein the first rotation lock comprises:
a body;
a plurality of ball pockets disposed in the body; and
a plurality of ball bearings disposed in the plurality of ball pockets, respectively.

7. The rod bending system of claim 6, wherein during a rod insertion of the rod, the plurality of ball bearings undergo ball translation radially outwards in each of the plurality of ball pockets.

8. The rod bending system of claim 7, wherein the plurality of ball bearings prevent a rod rotation of the rod with respect to the first rotation lock.

9. The rod bending system of claim 1, wherein the second lock collar further comprises a second rotation lock.

10. The rod bending system of claim 1, wherein the first linear lock is oriented to limit a rod removal of the rod.

11. The rod bending system of claim 10, wherein the second linear lock is oriented to limit a rod insertion of the rod.

12. The rod bending system of claim 11, wherein the first lock collar is used to cause the rod insertion of the rod.

13. The rod bending system of claim 12, wherein the second lock collar is used to cause the rod removal of the rod.

14. The rod bending system of claim 13, wherein the first lock collar is used to cause rod rotation of the rod.

15. A rod bending system, comprising:
a cart; and
a sterile cartridge disposed on the cart, comprising:
a bender configured to bend a rod;
a positioner;
a carriage configured to hold the rod;
a rotate shaft coupled to the carriage and the positioner; and
a feed screw coupled to the carriage and the positioner.

16. The rod bending system claim 15, wherein the positioner comprises:
a first drive shaft coupled to the rotate shaft and configured to cause a rotation of the rotate shaft.

17. The rod bending system of claim 16, wherein the rotation of the rotate shaft causes the rod to rotate.

18. The rod bending system claim 15, wherein the positioner comprises:
a second drive shaft coupled to the feed screw and configured to cause a rotation of the feed screw.

19. The rod bending system of claim 18, wherein the rotation of the feed screw causes the carriage and the rod to translate.

20. The rod bending system of claim 19, wherein the rod is configured to translate through the positioner to contact the bender.
